(19) United States
(12) Patent Application Publication
(10) Pub. No.: US 2022/0363225 A1
(43) Pub. Date: Nov. 17, 2022

(54) Vehicular cart treatment method and apparatus

(71) Applicant: Thomas George Podell, South Bend, IN (US)

(72) Inventor: Thomas George Podell, South Bend, IN (US)

(21) Appl. No.: 17/300,294

(22) Filed: May 13, 2021

(51) Int. Cl. B60S 3/04; B60P 3/14; B60P 1/43; A61L 2/26; A61L 2/22

(52) U.S. Cl. CPC B60S 3/04; B60P 3/14; B60P 1/435; A61L 2/26; A61L 2/22; G09F 21/048

(57) ABSTRACT

A towable trailer vehicle is provided having a pass-through cart treatment chamber formed from lateral side to side between two ramp doors. The chamber forms a spray tunnel in which carts are moved by a conveyor from various treatment stations to the next, and which can include cleaning, sanitizing and/or disinfecting, rinsing, drying, and applying protective coatings to each cart. The various fluids used in the spray treatment are stored, prepared, controlled, and collected for reuse and/or disposal within the trailer. The trailer is dimensioned to allow sufficient advertising, information, and/or aesthetic decor thereon to facilitate its use and desirability. The trailer structure provides apparatus security during periods of non-use.

### BACKGROUND OF THE INVENTION

The present invention relates generally to portable apparatus for treating carts, and more particularly, to vehicles which can be used to clean, disinfect, sanitize, and/or coat shopping carts and the like on-site and/or on a temporary basis.

Shopping carts and the like are known to be a substantial source of microbial contact with humans. By “carts,” the present application refers to not just shopping carts, per se, but also similarly situated items, such as meat carts, bakery carts, industrial carts, baskets, wheel chairs, portable tables, certain dunnage, frames, dollies, at the like which come into contact with a variety of persons and/or biological materials, such that microbial residue is deposited on the cart in the normal course of use and can be thereafter transmitted to different persons having contact with the same cart.

At present, several methods of reducing the likelihood of transmission of disease from subsequent cart use have been employed. For example, grocery stores have employed masked persons to stand beside cart racks to personally spray and/or wipe down specific shopping cart surfaces, sequentially on individual carts, which are the likely points of contact between subsequent uses on each shopping cart. Such methods do not completely remove microbial deposits on the carts and are labor intensive and of inconsistent treatment quality.

Automated cart treatment apparatus have been suggested, such as a pass-through arch under which the carts are pushed for treatment by infra-red or other radiant energy devices. However, the effectiveness of commercially viable radiant energy treatments to destroy harmful microbes is greatly reduced by the distance of the cart surface from the energy source. Thus, upper cart handle surfaces, passing closer to arch, may receive substantially greater “disinfectant” treatment than the more distant surfaces which children who are placed in the cart will typically come into contact with. Further, use of such arches has been commonly limited to locations outside of the store and exposed to the exterior environment, which can adversely affect treatment value according, for example, to localized weather conditions. Also, such arches typically have a limited “tunnel” dimension, and do not lend themselves to application of more efficient spray treatments, especially since there is no shielding from fluid dissipation to the immediate environment, or collection of fluid residue out of the arch. Further, these arch apparatus are less portable than would be preferred, and may need special and expensive security for continued on-site placement exterior of a building, especially during non-use, after business hours.

Automated vehicular cart treatment has been suggested to provide greater portability and inherent security during non-use, such as a containment chamber formed for longitudinal entry and exit from a towable vehicle, wherein a single cart or limited number of carts are placed within the then-sealed chamber inside of the vehicle and then subjected to a individual or batch spray wash treatment. While this allows the treatment fluid to be efficiently managed and more consistent treatment of the overall cart, it is inherently slower than a pass-through treatment process, and can be more labor intensive, since even batch treatment typically requires manual movement of the carts, at least in and out of the chamber, and opening and closing the chamber doors between each treatment session.

### OBJECTIVES OF THE INVENTION

Accordingly, a primary objective of the invention is to provide an improved method of and apparatus for treatment of carts within a vehicle. These improvements include providing such arrangements which:
a. are inexpensive to manufacture, install, maintain, and repair,
b. increase cart throughput in the treatment process,
c. minimize labor resources needed for operation,
d. accommodate a variety of different carts,
e. increase accessibility and user convenience,
f. minimize environmental impact from apparatus use, and
g. facilitate the aesthetic appeal of the apparatus and increase consumer confidence in and desirability for the apparatus.

### SUMMARY OF THE INVENTION

These and other objectives of the present invention are achieved by the provision of a towable trailer vehicle having a pass-through cart treatment chamber formed from lateral side to side between two ramp doors. The chamber forms a spray tunnel in which carts are moved by a conveyor from various treatment stations to the next, and which can include cleaning, sanitizing and/or disinfecting, rinsing, drying, and applying protective coatings to each cart. The various fluids used in the spray treatment are stored, prepared, controlled, and collected for reuse and/or disposal within the trailer. The trailer is dimensioned to allow sufficient advertising, information, and/or aesthetic decor thereon to facilitate its use and desirability. The trailer structure provides apparatus security during periods of non-use.

Other objects, advantages, and novel features of the present invention will become readily apparent from the following drawings and detailed description of certain preferred and alternative embodiments.

### BRIEF DESCRIPTION OF THE DRAWINGS:

FIG. 1 shows a right side view of a vehicle incorporating the present invention.

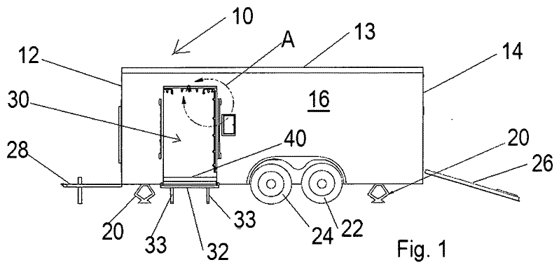

FIG. 2 shows an upper right, front perspective view of the vehicle of FIG. 1 with the roof removed.

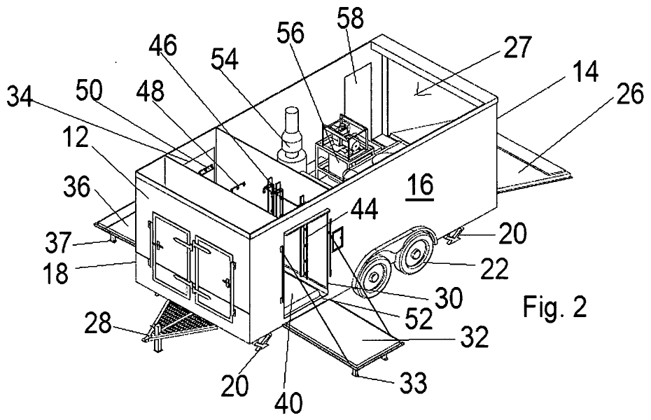

FIG. 3 shows an enlarge view of area A in FIG. 1 with more nozzle detail.

FIG. 4 shows a top view of the vehicle of FIG. 1 with the roof removed.

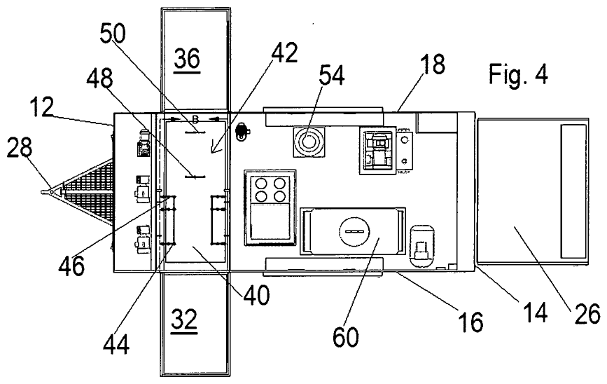

FIG. 5 shows a rear view of the vehicle of FIG. 1.

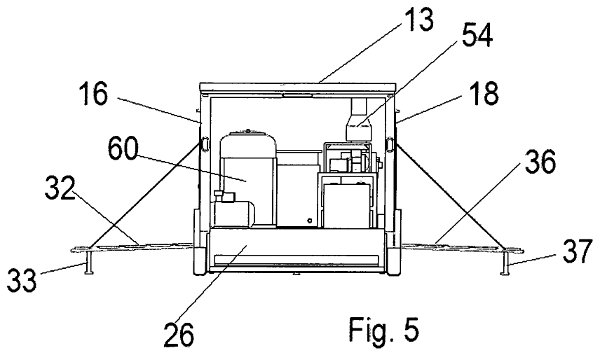

### DETAILED DESCRIPTION OF PREFERRED EMBODIMENTS:

The figures show schematically, and in best mode versions, embodiments of the present invention, as applied to towable trailer-type of vehicle, both generically and specifically. The drawings are not to scale from one view to the next.

The following numbered and lettered features are illustrated in the drawings:

Vehicle 10, having:
a front 12,
a roof 13,
a rear 14,
a first side 16, and
a second side 18,

such that the vehicle is longitudinally extending from front 12 to rear 14, and laterally extending from first side 16 to second side 18, and side 16 and 18 are opposing each other.

Vehicle 10 also includes various other conventional features, such as:
stabilizing jacks 20,
wheels 22 and 24,
a rear ramp door 26 for selectively closing a rear opening 27, and
a bumper-pull hitch 28 for connection to a conventional motorized vehicle (not shown).

Vehicle 10 further includes features specially used in the present invention, such as:
first opening 30 in first side 16,
side ramp door 32 for selectively closing opening 30,
door feet 33 for elevating ramp door 32,
second opening 34 in second side 18,
side ramp door 36 for selectively closing opening 34,
door feet 37 for elevating ramp door 36, and
conveyor 40, preferably movable from opening 30 to opening 34 on its upper surface,

such that treatment chamber 42 is formed between openings 30 and 34, having mounted therein various spraying apparatus for dispensing fluid upon carts inserted through opening 30.

In especially preferred embodiments, those spraying apparatus include:
washing sprays 44 to clean the carts,
disinfecting sprays 46 to sanitize the carts,
rinsing sprays 48, and
misting sprays 40 to apply protective coatings,

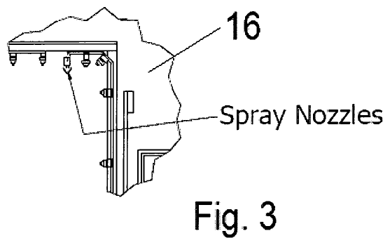

preferably such that the spraying apparatus are fixedly mounted to the interior walls of chamber 42 and each includes a plurality of spray nozzles arranged to cause fluid impingement upon said carts form a plurality of different directions, especially as shown in FIG. 3 with reference to individual spray nozzles. A supplemental or auxiliary spraying device (not shown), such as a conventional spray wand, is provided, preferably in fluid connection with the washing sprays, for dispensing fluid upon said carts within or outside of said chamber.

Preferred embodiments can also include a blow-off stage after application of the rinsing sprays and/or misting sprays within chamber 24 in order to facilitate drying, by means of conventional heated or ambient air blowers (not shown) to remove standing water or fluid from the cart surfaces.

Vehicle 10 also includes apparatus for collecting fluid run-off from the carts after the spray treating, including, for example, one or more movable catch pans 52, mounted under conveyor 40 and selectively extendable to the vehicle exterior under ramps 32 and 36 as needed. Fluid collected therein is preferably pumped back into the vehicle for processing, filtering, reuse, and/or disposal.

Various apparatus 54 and 56, as desired for given applications are also provided in vehicle 10, for heating, pressurizing, mixing, preparing and treating the various fluids used in the treatment process, and mounted at locations within said vehicle and longitudinally spaced within said vehicle outside of said chamber. Similarly, various reservoirs 60 are provided in vehicle 10, such as for fluid to be provided to the spray apparatus and collected as run-off therefrom, and those reservoirs are mounted at locations within the vehicle and longitudinally spaced within the vehicle outside of said chamber. Typically, rear opening 27 provides access for servicing and control of these various apparatus, with ramp door 26 providing security against unauthorized access thereto. However, a supplemental door 58 can be used for additional access in preferred embodiments.

When ramp doors 32 and 36 are lowered to give access to the side openings, carts to be treated can be placed on those doors and guided into and out of chamber 42. In that sense, ramp doors 32 and 36 can serve as guide tracks for the carts passing through chamber 42. In certain embodiments, door feet 33 can be raised or formed high enough to allow ramp door 32 to provide a downward incline toward opening 30 so as to urge carts placed thereon to roll toward opening 30, and then onto conveyor 40 for additional motion through chamber 42. Similarly, door feet 37 can be lowered or formed low enough to allow ramp door 36 to provide an downward incline away from opening 34 so as to urge carts emerging from chamber 42 to then roll away from opening 34.

Preferably, at least one of sides 16 or 18 of vehicle 10 is formed with sufficient vertical and longitudinal dimension to support placement of readily visible advertising signage, such as indicating the purpose of the vehicle and advantages of its use. Alternatively, or additionally, sides 16 and/or 18 are dimensioned to support aesthetically desirable decorative treatment of vehicle 10 for its environment of usage.

Although the present invention has been shown and described herein with respect to certain preferred embodiments and alternative configurations, those were by way of illustration and example only. For example, the present invention has been shown in embodiments of a towable trailer. However, it will now be readily understood that this invention can be alternatively embodied in a motorized vehicle. Further, various different treatment stages can be also used in connection with the spray apparatus contemplated by the present invention. Accordingly, the spirit and scope of the present invention is intended to be limited only by the terms of the appended claims.

What is claimed is:

1. A vehicle for pass-through treatment of carts, comprising:
a longitudinally extending vehicle having a front, a rear, and first and second opposing sides,
first and second openings, respectively, in the first and second opposing sides, each of sufficient dimension to receive therethrough carts for treatment within the vehicle by passing through those openings,
a treatment chamber formed within said vehicle and between said first and second openings for performing pre-determined dispensing operations on carts to be inserted into said vehicle.

2. The vehicle according to claim 1 wherein said chamber includes spraying apparatus for dispensing fluid upon said carts as said carts pass through said chamber.

3. The vehicle according to claim 2 wherein said spraying apparatus includes a plurality of spray nozzles arranged to cause fluid impingement upon said carts form a plurality of different directions.

4. The vehicle according to claim 3 wherein said vehicle includes means for collecting fluid run-off from said carts and processing that fluid and matter entrained therewith for reuse and/or disposal.

5. The vehicle according to claim 3 wherein said dispensing operations include in sequence, spraying fluid to first wash said carts, then to sanitize said carts by disinfectant, then to rinse said carts, then to dry said carts, and then to apply a protective coating to said carts.

6. The vehicle according to claim 5 wherein the vehicle includes apparatus, for heating and pressurizing fluid to be applied through said spray apparatus, mounted at locations within said vehicle and longitudinally spaced within said vehicle outside of said chamber.

7. The vehicle according to claim 6 wherein the vehicle includes reservoirs for fluid to be provided to the spray apparatus and collected as run-off therefrom, said reservoirs being mounted at locations within said vehicle and longitudinally spaced within said vehicle outside of said chamber.

8. The vehicle according to claim 7 wherein the vehicle is provided with a rear ramp door for accessing apparatus or reservoirs therein which are outside of said chamber.

9. The vehicle according to claim 2 wherein ramp doors are mounted to selectively close each of said first and second openings, as well to provide a guide track for carts passing through said first and second openings when the ramps are not completely closing said first and second openings.

10. The vehicle according to claim 9 wherein the ramp door associated with the first opening can be disposed at a downward incline toward said vehicle, so as to urge carts placed thereon to roll toward said first opening, and the ramp door associated with said second opening can be disposed at a downward incline away from said vehicle, so as to urge carts placed therein to roll away from said second opening.

11. The vehicle according to claim 2 wherein a conveyor is mounted within said chamber to facilitate movement of said carts between the first and second openings.

12. The vehicle according to claim 2 wherein said spraying apparatus is fixedly mounted to said chamber, and an auxiliary spraying device is provided for dispensing fluid upon said carts within or outside of said chamber.

13. The vehicle according to claim 2 wherein the vehicle is formed as a towable trailer having a hitch for connection to a motorized vehicle.

14. The vehicle according to claim 13 wherein at one of said sides is formed with sufficient vertical and longitudinal dimension to support placement of advertising signage indicating the purpose of the vehicle and advantages of its use.
